(19) United States
(12) Patent Application Publication
(10) Pub. No.: US 2013/0317089 A1
(43) Pub. Date: Nov. 28, 2013

(54) Compositions and Methods Useful for Treatment and Prevention of Incontinence

(71) Applicants: John H. Wolfe, Blue Bell, PA (US); Rita Valentino, Villanova, PA (US)

(72) Inventors: John H. Wolfe, Blue Bell, PA (US); Rita Valentino, Villanova, PA (US)

(21) Appl. No.: 13/885,057

(22) PCT Filed: Nov. 10, 2011
(86) PCT No.: PCT/US11/60132

§ 371 Date: Aug. 13, 2013

Related U.S. Application Data
(60) Provisional application No. 61/413,274, filed on Nov. 12, 2010.

(51) Int. Cl. A61K 48/00; A61K 45/06

(52) U.S. Cl. CPC A61K 48/0058; A61K 45/06
USPC 514/44 R; 435/320.1

(57) ABSTRACT

Compositions and methods effective for increasing bladder control are provided.

This application claims priority to U.S. Provisional Application 61/413,274 filed Nov. 12, 2010, the entire contents being incorporated herein by reference as though set forth in full.

Pursuant to 35 U.S.C. §202(c) it is acknowledged that the U.S. Government has rights in the invention described, which was made in part with funds from the National Institutes of Health, Grant Number P50-DK052620.

### FIELD OF THE INVENTION

This invention relates to the fields of molecular biology and bladder control. More specifically, the invention provides compositions and methods useful of inhibiting incontinence in patients in need thereof.

### BACKGROUND OF THE INVENTION

Several publications and patent documents are cited through the specification in order to describe the state of the art to which this invention pertains. Each of these citations is incorporated herein by reference as though set forth in full.

Barrington's nucleus, mediates the descending limb of the micturition reflex through its Forward Reverse projections to lumbosacral spinal preganglionic neurons that provide parasympathetic input to the bladder. This spinal pathway activates the parasympathetic input to the bladder that is responsible for contraction when bladder pressure reaches micturition threshold. Many Barrington's nucleus neurons also project to the major norepinephrine nucleus, the locus coeruleus (LC). Excitation of LC neurons causes arousal through LC projections to the cortex. Through its spinal and LC projections, Barrington's nucleus is positioned to coordinate the descending limb of the micturition reflex with a central limb that is responsible for interrupting ongoing behavior and facilitating voiding-related behaviors (FIG. 1). Many Barrington's nucleus neurons that project to both the LC and spinal cord express the stress-related neuropeptide, corticotropin-releasing factor (CRF). CRF excites LC neurons and can produce cortical electroencephalographic indices of arousal through this pathway. Physiological studies suggest that CRF in projections from Barrington's nucleus to the spinal cord has an inhibitory influence on micturition. By facilitating arousal and inhibiting bladder contraction, CRF in this circuit may help to maintain continence.

Over 13 million American men and women of all ages suffer from urinary incontinence. The social implications for an incontinent patient include loss of self-esteem, embarrassment, restriction of social and sexual activities, isolation, depression and, in some instances, dependence on caregivers. Incontinence is the most common reason for institutionalization of the elderly.

Incontinence may occur when the muscles of the urinary system malfunction or are weakened. Other factors, such as trauma to the urethral area, neurological injury, hormonal imbalance or medication side-effects, may also cause or contribute to incontinence. There are five basic types of incontinence: stress incontinence, urge incontinence, mixed incontinence, overflow incontinence and functional incontinence. Stress urinary incontinence (SUI) is the involuntary loss of urine that occurs due to sudden increases in intra-abdominal pressure resulting from activities such as coughing, sneezing, lifting, straining, exercise and, in severe cases, even simply changing body position. Urge incontinence, also termed “hyperactive bladder” “frequency/urgency syndrome” or “irritable bladder,” occurs when an individual experiences the immediate need to urinate and loses bladder control before reaching the toilet. Mixed incontinence is the most common form of urinary incontinence. Inappropriate bladder contractions and weakened sphincter muscles usually cause this type of incontinence. Mixed incontinence is a combination of the symptoms for both stress and urge incontinence. Overflow incontinence is a constant dripping or leakage of urine caused by an overfilled bladder. Functional incontinence results when a person has difficulty moving from one place to another. It is generally caused by factors outside the lower urinary tract, such as deficits in physical function and/or cognitive function.

Clearly a need exists for composition and methods which prevent or inhibit incontinence.

### SUMMARY OF THE INVENTION

In accordance with the present invention, a vector comprising a corticotropin-releasing factor (CRF) encoding nucleic acid is provided. In a preferred embodiment, the vector is an adeno-associated viral vector suitable for administration to humans. In another aspect, the CRF encoding nucleic acid is operably linked to a promoter which functions in Barrington's nucleus. Also provided is a composition comprising the vector described above in a pharmaceutically acceptable carrier.

In yet another embodiment of the invention, a method of treating urinary incontinence in a subject in need of such treatment is disclosed. An exemplary method entails administering to said subject an effective amount of the CRF encoding composition, the amount being effective to inhibit or prevent incontinence. Urinary incontinence to be treated includes urge incontinence, stress incontinence, mixed urge/stress incontinence or neurogenic incontinence.

### BRIEF DESCRIPTION OF THE DRAWINGS

FIG. 1. A schematic diagram of central and visceral effects of Barrington's nucleus activation.

FIG. 2. A schematic of the methods used to construct the AAV vector of the invention.

FIG. 3. Transduction of Barrington's nucleus neurons.

FIG. 4. Comparison of CRF-immunoreactive neurons in Barrington's nucleus of a rat that was injected with AAV-1 expressing CRF cDNA, AAV-1 expressing the reverse cDNA and a case in which no virus was injected.

FIG. 5. Fluorescent photomicrographs at the level of the lumbosacral spinal cord of rats that were injected with AAV-1 expressing CRF cDNA or the reverse cDNA sequence in Barrington's nucleus.

FIG. 6. The effects of CRF expression on urodynamics is shown. A) The results of in vivo cystometry are shown. B) Graph of the results for the cystometry procedure shown in A.

FIG. 7. FIG. 7A. Fluorescent photomicrographs of CRF innervation of the LC in rats injected with AAV-1 expressing CRF cDNA or AAV-1 expressing the reverse sequence. FIG. 7B A bar graphs showing the results of CRF expression on burying behavior.

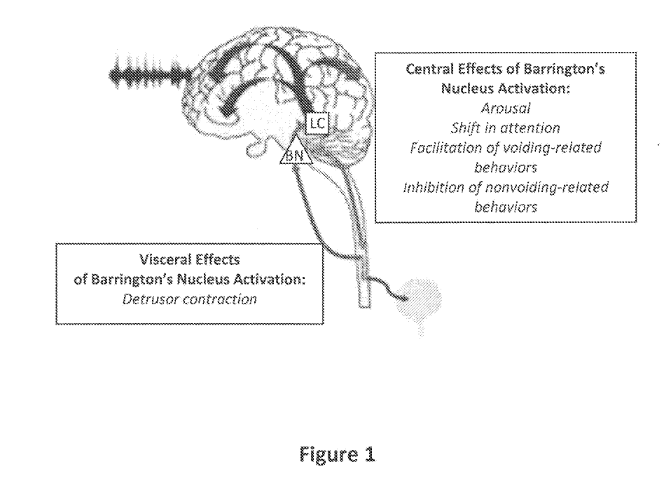

### DETAILED DESCRIPTION OF THE INVENTION

In accordance with the present invention, an adeno-associated viral vector was developed which expresses cortico-releasing factor (CRF) in a subset of brain cells, i.e., Barrington's nucleus. Exogenous expression of CRF in these cells results in bladder contraction and retention of urine. Such compositions are useful for the treatment and control of incontinence.

### I. DEFINITIONS

The following definitions are provided to facilitate an understanding of the present invention. Unless defined otherwise, all technical and scientific terms used herein have the same meaning as commonly understood by one of ordinary skill in the art to which this invention belongs. Generally, conventional methods of molecular biology, microbiology, recombinant DNA techniques, cell biology, and virology within the skill of the art are employed in the present invention. Such techniques are explained fully in the literature, see, e.g., Maniatis, Fritsch & Sambrook, Molecular Cloning: A Laboratory Manual (1982); DNA Cloning: A Practical Approach, Volumes I and II (D. N. Glover, ed. 1985); Oligonucleotide Synthesis (M. J. Gait, ed. 1984); Nucleic Acid Hybridization (B. D. Hames & S. J. Higgins, eds. (1984)); Animal Cell Culture (R. I. Freshney, ed. 1986); and RNA Viruses: A Practical Approach, (Alan, J. Cann, Ed., Oxford University Press, 2000).

For purposes of the invention, “Nucleic acid”, “nucleotide sequence” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. Alternatively, this term may refer to a DNA that has been sufficiently separated from (e.g., substantially free of) other cellular components with which it would naturally be associated.

Corticotropin-releasing hormone (CRH), originally named corticotropin-releasing factor (CRF), and also called corticoliberin, is a polypeptide hormone and neurotransmitter involved in the stress response. It belongs to corticotropin-releasing factor family. Corticotropin-releasing hormone (CRH) is a 41-amino acid peptide derived from a 191-amino acid preprohormone. CRH is secreted by the paraventricular nucleus (PVN) of the hypothalamus in response to stress. The amino acid sequence is SQEPPISLDLTFHLLREVLEMTKADQLAQQAHSNRKLLDIA (SEQ ID NO: 1).

“Isolated” is not meant to exclude artificial or synthetic mixtures with other compounds or materials, or the presence of impurities that do not interfere with the fundamental activity, and that may be present, for example, due to incomplete purification. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

According to the present invention, an isolated or biologically pure molecule or cell is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The term “promoter” or “promoter region” generally refers to the transcriptional regulatory regions of a gene. The “promoter region” may be found at the 5′ or 3′ side of the coding region, or within the coding region, or within introns. Typically, the “promoter region” is a nucleic acid sequence which is usually found upstream (5′) to a coding sequence and which directs transcription of the nucleic acid sequence into mRNA. The “promoter region” typically provides a recognition site for RNA polymerase and the other factors necessary for proper initiation of transcription.

Promoters useful in some embodiments of the present invention may be tissue-specific or cell-specific. The term “tissue-specific” as it applies to a promoter refers to a promoter that is capable of directing selective expression of a nucleotide sequence of interest to a specific type of tissue in the relative absence of expression of the same nucleotide sequence of interest in a different type of tissue (e.g., liver). The term “cell-specific” as applied to a promoter refers to a promoter which is capable of directing selective expression of a nucleotide sequence of interest in a specific type of cell in the relative absence of expression of the same nucleotide sequence of interest in a different type of cell within the same tissue (see, e.g., Higashibata, et al., J. Bone Miner. Res. January 19(1):78-88 (2004); Hoggatt, et al., Circ. Res., December 91(12):1151-59 (2002); Sohal, et al., Circ. Res. July 89(1):20-25 (2001); and Zhang, et al., Genome Res. January 14(1):79-89 (2004)). The term “cell-specific” when applied to a promoter also means a promoter capable of promoting selective expression of a nucleotide sequence of interest in a region within a single tissue. Alternatively, promoters may be constitutive or regulatable. Additionally, promoters may be modified so as to possess different specificities.

The term “vector” relates to a single or double stranded circular nucleic acid molecule that can be infected, transfected or transformed into cells and replicate independently or within the host cell genome. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. An “expression vector” is a specialized vector that contains a gene or nucleic acid sequence with the necessary regulatory regions needed for expression in a host cell. The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

As used herein, “pharmaceutical formulations” include formulations for human and veterinary use which exhibit no significant adverse toxicological effect. The phrase “pharmaceutically acceptable formulation” as used herein refers to a composition or formulation that allows for the effective distribution of the nucleic acid molecules of the instant invention in the physical location most suitable for their desired activity. The term “pharmaceutically acceptable” means that the carrier can be taken into the subject with no significant adverse toxicological effects on the subject.

The term “therapeutically effective amount” is the amount present that is delivered to a subject to provide the desired physiological response. Methods for preparing pharmaceutical compositions are within the skill in the art, for example as described in Remington's Pharmaceutical Science, 18th ed., Mack Publishing Company, Easton, Pa. (1990), and The Science and Practice of Pharmacy, 2003, Gennaro et al.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3rd Ed.), American Pharmaceutical Association, Washington, 1999.

The term “treating” or “to treat” as used herein means activity resulting in the prevention, reduction, partial or complete alleviation or cure of a disease or disorder. The term “modulate” means altering (i.e., increasing or decreasing) the biological activity of a system. Activity can be modulated by a variety of mechanisms such as modifying expression levels of CRF in Barrington's nucleus through introduction of exogenous vectors encoding the same.

A “subject” or “patient” includes, but is not limited to animals, including mammalian species such as murine, porcine, ovine, bovine, canine, feline, equine, human, and other primates.

The term “kit” refers to a combination of reagents and other materials.

### II. THERAPEUTIC USES OF THE VECTOR CONSTRUCTS OF THE INVENTION

The CRF encoding vector constructs may be used according to this invention, for example, as therapeutic agents that enhance bladder control. In a preferred embodiment of the present invention, the vector contructs can be administered to a patient via infusion in a biologically compatible carrier. The constructs may be administered alone or in combination with other agents known to decrease incontinence. An appropriate composition in which to deliver the AAV vector may be determined by a medical practitioner upon consideration of a variety of physiological variables as contemplated hereinbelow. A variety of compositions well suited for different applications and routes of administration are well known in the art and are described hereinbelow.

In a preferred embodiment of the invention, the expression vector comprising nucleic acid sequences encoding CRF is a viral vector. Viral vectors which may be used in the present invention include, but are not limited to, adenoviral vectors (with or without tissue specific promoters/enhancers), adeno-associated virus (AAV) vectors of multiple serotypes (e.g., AAV-1-9) and recombinant AAV vectors, lentivirus vectors and pseudo-typed lentivirus vectors (e.g., Ebola virus, vesicular stomatitis virus (VSV), and feline immunodeficiency virus (FIV)), herpes simplex virus vectors, vaccinia virus vectors, and retroviral vectors. In preferred embodiments, rAAV-2 will be used in in vitro assays, rAAV-8 will be used in mouse studies and rAAV-6, 8, or 9 will be used as a carrier for in vivo administration of the CRF of the invention to primates.

For some applications, an expression construct may further comprise regulatory elements which serve to drive expression in a particular cell or tissue type. Such regulatory elements are known to those of skill in the art and discussed in depth in Sambrook et al. (1989) and Ausubel et al. (1992). The incorporation of tissue specific regulatory elements in the expression constructs of the present invention provides for at least partial tissue tropism for CRF expression. For example, the CRF encoding constructs can be subcloned into a vector downstream of a tissue (i.e., neuronal) specific promoter/enhancer to treat incontinence. Additionally, polyadenylation sequences can be inserted downstream of the CRF encoding nucleic acid.

### III. PHARMACEUTICAL COMPOSITIONS

The expression vectors of the present invention may be incorporated into pharmaceutical compositions that may be delivered to a subject. In a particular embodiment of the present invention, pharmaceutical compositions comprising isolated nucleic acids which enable the recipient to produce therapeutically effective levels of CRF that modulate incontinence in the recipient are provided. The compositions may be administered alone or in combination with at least one other agent, such as a stabilizing compound, which may be administered in any sterile, biocompatible pharmaceutical carrier, including, but not limited to, saline, buffered saline, dextrose, and water. In preferred embodiments, the pharmaceutical compositions also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., 18th Edition, Easton, Pa. (1990).

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions. The pharmaceutical compositions of the present invention may be manufactured in any manner known in the art (e.g., by means of conventional mixing, dissolving, granulating, dragee-making, levigating, emulsifying, encapsulating, entrapping, or lyophilizing processes).

After pharmaceutical compositions have been prepared, they may be placed in an appropriate container or kit and labeled for treatment. For administration of CRF expression vectors, such labeling would include amount, frequency, and method of administration.

Pharmaceutical compositions suitable for use in the invention include compositions wherein the constructs are contained in an effective amount to achieve the intended therapeutic purpose. Determining a therapeutically effective dose is well within the capability of a skilled medical practitioner using the techniques provided hereinbelow. Therapeutic doses will depend on, among other factors, the age and general condition of the subject, the severity of the incontinence, and the strength of the control sequences regulating the expression levels of CRF. Thus, a therapeutically effective amount in humans will fall in a relatively broad range that may be determined by a medical practitioner based on the response of an individual.

### IV. METHODS OF TREATMENT AND DELIVERY

Nucleic acids encoding CRF either in plasmid or viral vector forms alone or in combination with other agents, may be directly infused into a patient in an appropriate biological carrier, preferably by IV administration. One of skill in the art could readily determine specific protocols for using the constructs of the present invention for the therapeutic treatment of a particular patient. In this regard, the compositions may be delivered subcutaneously, epidermally, intradermally, intracranially, intrathecally, intraorbitally, intramucosally, intraperitoneally, intravenously, intraarterially, orally, intrahepatically or intramuscularly. Other modes of administration include oral and pulmonary administration, suppositories, and transdermal applications.

Dosage levels on the order of about 1 μg/kg to 100 mg/kg of body weight per administration are useful in the treatment of incontinence. In regard to dosage, the constructs can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of the construct per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of the construct per kg of bodyweight. Alternatively AAV doses can be determined using vectors genomes/kg, which in turn can be converted to μg capsid protein/kg. The unit dose, for example, can be administered by injection for example, intravenous, intramuscular, intrathecally, or directly into a tissue such as the brain.

One skilled in the art can also readily determine an appropriate dosage regimen for administering the constructs of the invention to a given subject. For example, the CRF encoding constructs can be administered to the subject once, e.g., as a single injection or deposition at Barrington's nucleus. Alternatively, the vectors can be administered multiple times to a subject. However, this may require the use of alternative AAV vectors to avoid anti-AAV antibodies that may develop upon initial exposure to the rAAV. It may also be desirable to administer such vectors in conjunction with an immunosuppressive agent in order to suppress this undesired immune response. One skilled in the art will appreciate that the exact individual dosages may be adjusted somewhat depending on a variety of factors, including the specific construct being administered, the time of administration, the route of administration, the nature of the formulation, the rate of excretion, the particular infection being treated, the severity of the incontinence, the pharmacodynamics of the oligonucleotide agent, and the age, sex, weight, and general health of the patient. Wide variations in the necessary dosage level are to be expected in view of the differing efficiencies of the various routes of administration. For instance, oral administration generally would be expected to require higher dosage levels than administration by intravenous or intravitreal injection. Variations in these dosage levels can be adjusted using standard empirical routines of optimization, which are well-known in the art. Optimum dosages may vary depending on the relative potency of individual compounds, and can generally be estimated based on EC₅₀s found to be effective in in vitro and in vivo animal models. Changes in dosage may result and become apparent from the results of diagnostic assays. For example, the subject can be monitored after administering the composition. Based on information from the monitoring, an additional amount of the composition can be administered.

### V. KITS AND ARTICLES OF MANUFACTURE

Any of the aforementioned compositions or methods can be incorporated into a kit which may contain at least one CRF encoding vector. If the pharmaceutical composition in liquid form is under risk of being subjected to conditions which will compromise the stability of the vector, it may be preferred to produce the finished product containing the vectors in a solid form, e.g. as a freeze dried material, and store the product is such solid form. The product may then be reconstituted (e.g. dissolved or suspended) in a saline or in a buffered saline ready for use prior to administration.

Hence, the present invention provides a kit comprising (a) a first component containing the CRF encoding vectors as defined hereinabove, optionally in solid form, and (b) a second component containing saline or a buffer solution (e.g. buffered saline) adapted for reconstitution (e.g. dissolution or suspension) or delivery of said vector. Preferably said saline or buffered saline has a pH in the range of 4.0-8.5, and a molarity of 20-2000 mM. In a preferred embodiment the saline or buffered saline has a pH of 6.0-8.0 and a molarity of 100-500 mM. In a most preferred embodiment the saline or buffered saline has a pH of 7.0-8.0 and a molarity of 120-250 mM.

### VI. CLINICAL APPLICATIONS

As mentioned previously, a preferred embodiment of the invention comprises delivery of a CRF encoding vector to Barrington's nucleus to a patient in need thereof. Formulation, dosages and treatment schedules have also been described hereinabove. Phase I clinical trials can be designed to assess the safety, tolerability, pharmacokinetics, and pharmacodynamics of the vector constructs of the invention. These trials may be conducted in an inpatient clinic, where the subject suffering from an incontinence can be observed by full-time medical staff. After the initial safety of the therapy has been performed, Phase II trials can assess clinical efficacy of the therapy; as well as to continue Phase I assessments in a larger group of volunteers and patients. Subsequently, Phase III studies on large patient groups entail definitive assessment of the efficacy of the vector constructs for treatment of incontinence in comparison with current treatments. Finally, Phase IV trials involving the post-launch safety surveillance and ongoing technical support for the vector constructs can be completed.

The following materials and methods are provided to facilitate the practice of the invention.

### Vector Construction.

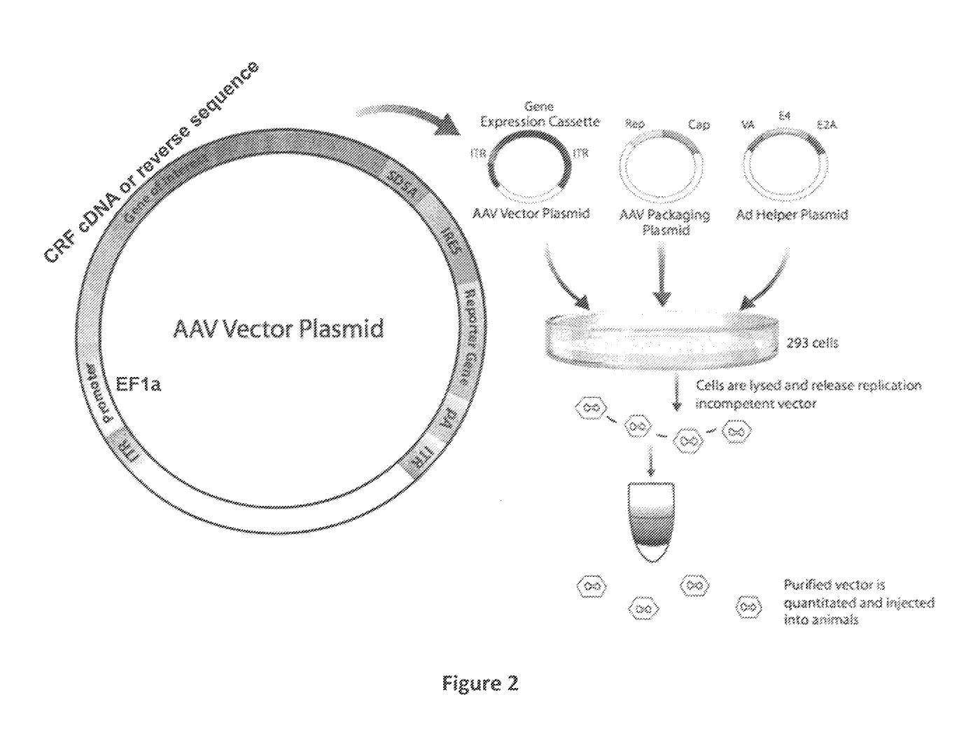

AAV-1 vectors were constructed that expressed either 1) CRF cDNA, 2) the reverse CRF cDNA sequence, or 3) green fluorescent protein (GFP) cDNA all driven by an EF1a promoter using methods previously described (Passini and Wolfe, J. Virol. (2001); 75(24): 12382-12392). See FIG. 2. Also included was the SV40 intron, WPRE and BGH polyA. Packaging, purification and determination of vector titers were performed by the University of Pennsylvania Vector Core. Recombinant vectors were purified using the CsCl sedimentation method and genome copy (GC) titers were determined. Injection titers were between 1.2 and 1.3×10¹³ GC/ml.

### Surgery.

Rats were anesthetized with isofluorane and positioned in a stereotaxic instrument for injection of a mixture of AAV-1 expressing GFP and AAV-1 expressing either CRF cDNA or the reverse cDNA sequence into Barrington's nucleus. Double barrel glass micropipettes were used to electrophysiologically localize Barrington's nucleus and inject 30-60 nl of the vector solution into the nucleus. Injections were made bilaterally. Four weeks after the injection rats were implanted with a catheter into the bladder for measurement of urodynamics using in vivo cystometry in the unanesthetized state.

### Behavior.

For some rats, behavior in a novel cage with 2 inches of bedding was videotaped and scored for the incidence of rearing, duration of grooming and duration of burying. Behavioral observation was done 24 h prior to surgery for cystometry.

### Cystometry.

Cystometry was performed in the unanesthetized state 24-48 h after implantation of the bladder catheter. The catheter was connected to tubing from a syringe pump which was in-line with a pressure transducer. Saline was infused into the bladder at a rate of 0.1 ml/min and bladder pressure and capacity were continuously monitored. Urine was collected in a pan that was situated on a scale below the cage so that the micturition volume could be continuously monitored for 1 h. Following cystometry, rats were transcardially perfused and brains and spinal cords were sectioned and stained to visualize CRF immunoreactivity.

The following examples illustrate certain embodiments of the invention. They are not intended to limit the scope of the invention in any way.

### Example I

### Gene Therapy for Incontinence

To better investigate the role of CRF in Barrington's nucleus projections to the LC and spinal cord, we used adeno-associated virus (AAV-1) vector-mediated transfer of CRF cDNA to increase CRF expression in rat Barrington's nucleus neurons. As a control, the reverse CRF cDNA sequence was used. Four weeks after injection of the vector into Barrington's nucleus, behavior and bladder urodynamics were assessed.

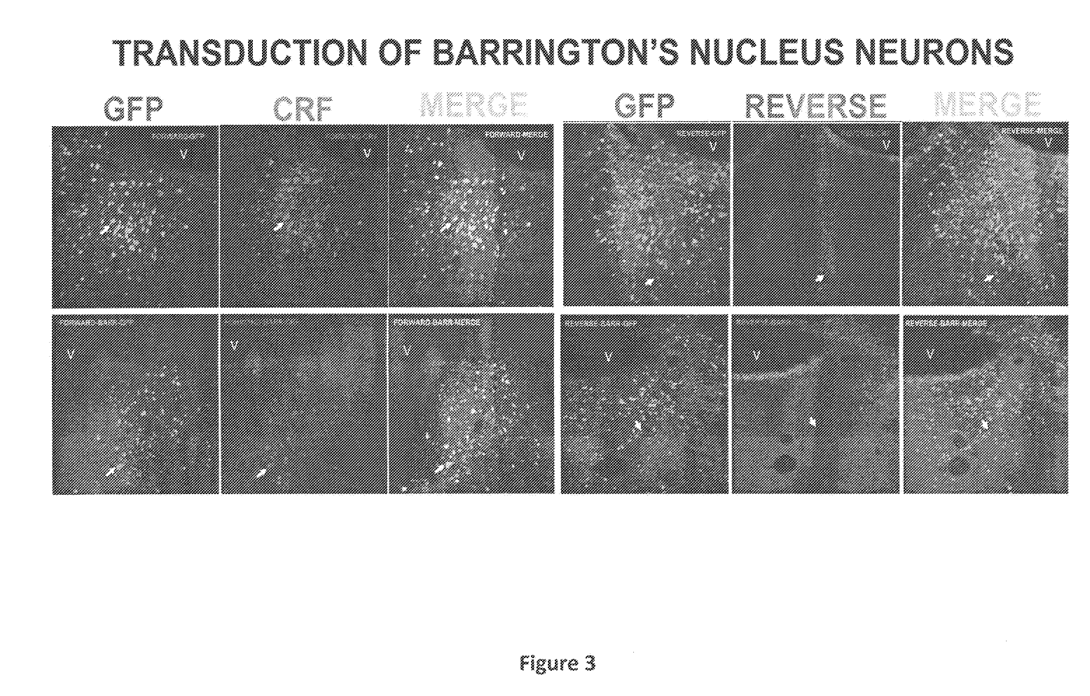

Fluoroscent photomicrographs of sections at the level of Barrington's nucleus showing cells expressing GFP, CRF-immunoreactivity and the merged image showing cells that express both GFP and CRF-immunoreactivity. See FIG. 3. The panels on the left and right were from rats that were injected with AAV-9 expressing CRF cDNA or the reverse cDNA sequence, respectively. All cases showed GFP labeled cells that were localized to the region of Barrington's nucleus. Because CRF is endogenously expressed by Barrington's nucleus neurons, CRF-immunoreactivity was present in Barrington's nucleus of all cases. However, cases injected with AAV-1 expressing CRF cDNA had many cells that were intensely CRF-immunoreactive. Arrows point to representative neurons for each case that express both GFP and CRF. V indicates the fourth ventricle. In total, 42 rats were injected bilaterally with either AAV-1 expressing CRF cDNA (n=25) or AAV-1 expressing the reverse CRF cDNA (n=17). Fourteen rats injected with AAV-1 expressing CRF cDNA had injections that were accurately localized bilaterally. The number of transduced Barrington's nucleus neurons per section as indicated by intensity ranged from 4-65 with a mean of 24+6. Comparisons between groups were done on sections that were identically processed and that were photographed with identical exposure times (100 ms). In some cases in which AAV-1 expressing CRF cDNA was injected, the intensity of CRF immunoreactivity was so great that additional photographs were taken at half the exposure time (50 ms).

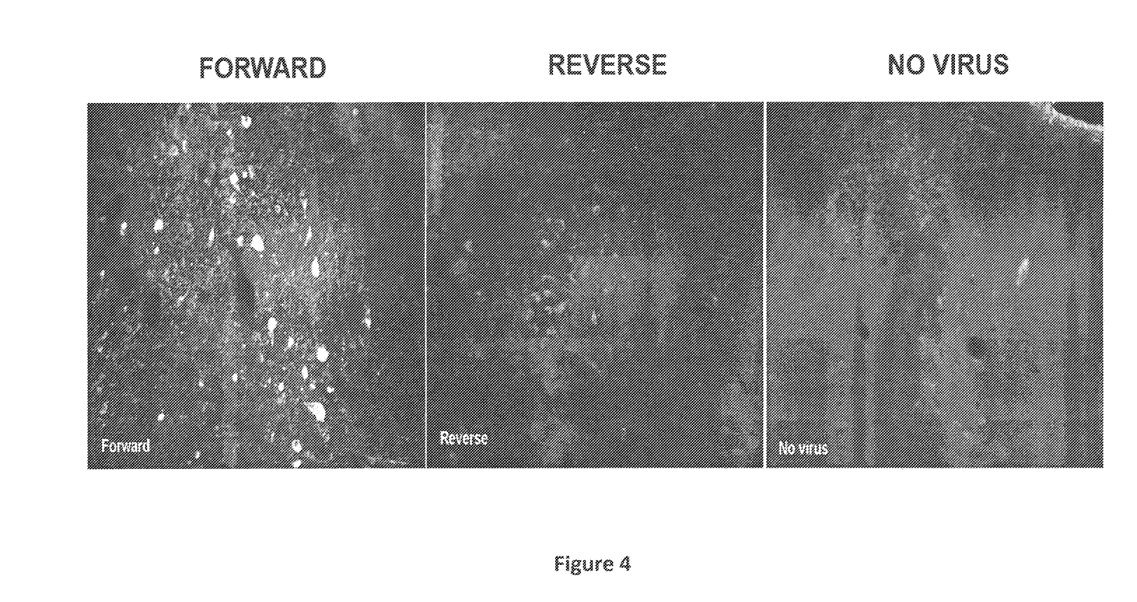

FIG. 4 shows a comparison of CRF-immunoreactive neurons in Barrington's nucleus of a rat that was injected with AAV-1 expressing CRF cDNA, AAV-1 expressing the reverse cDNA and a case in which no virus was injected. Sections were identically processed and photographed. Note that CRF-immunoreactivity in Barrington's nucleus is not compromised in cases in which the reverse sequence was injected compared to uninjected controls.

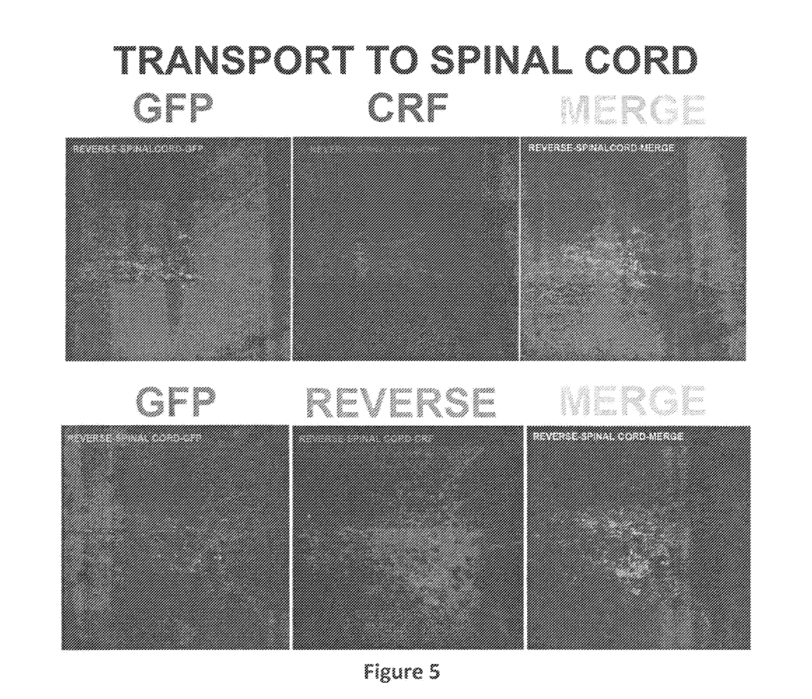

Fluorescent photomicrographs at the level of the lumbosacral spinal cord of rats that were injected with AAV-1 expressing CRF cDNA or the reverse cDNA sequence in Barrington's nucleus are shown in FIG. 5. CRF is typically present in the region that corresponds to the spinal preganglionic parasympathetic nucleus. The finding of GFP in the spinal preganglionic parasympathetic nucleus demonstrates transport of the GFP protein from Barrington's nucleus in the pons to the lumbosacral spinal cord. Localization of GFP in the same fibers as CRF (yellow in merged image) indicates that it was transported by CRF containing neurons of Barrington's nucleus. The spinal sections shown are from the same cases as those shown in the bottom panel of the Barrington's nucleus in FIG. 4.

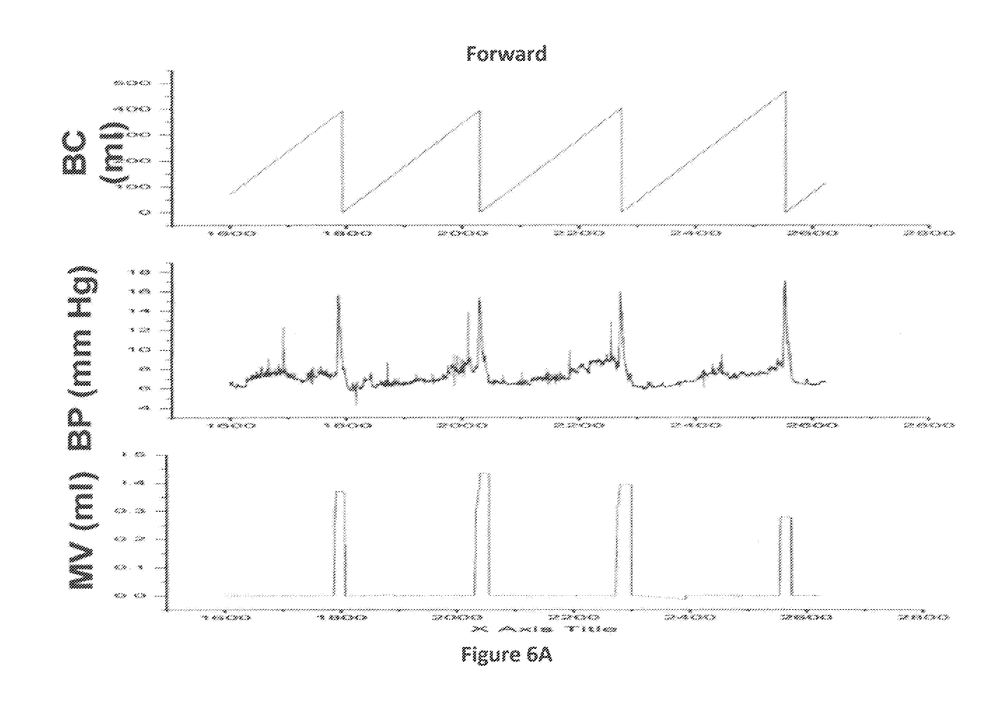

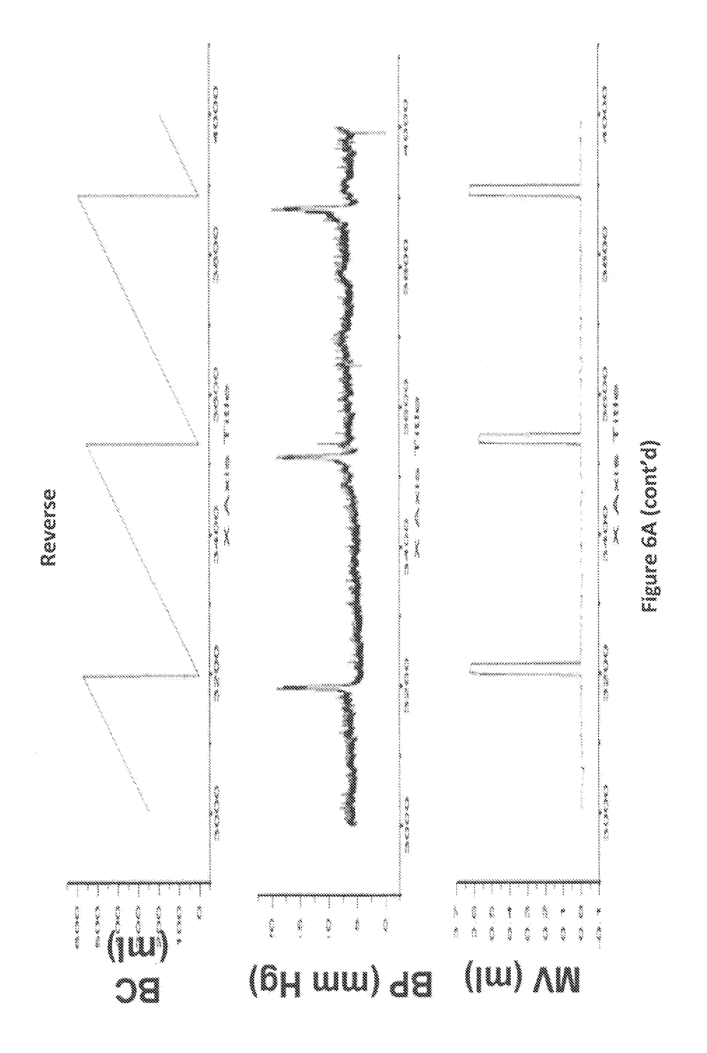

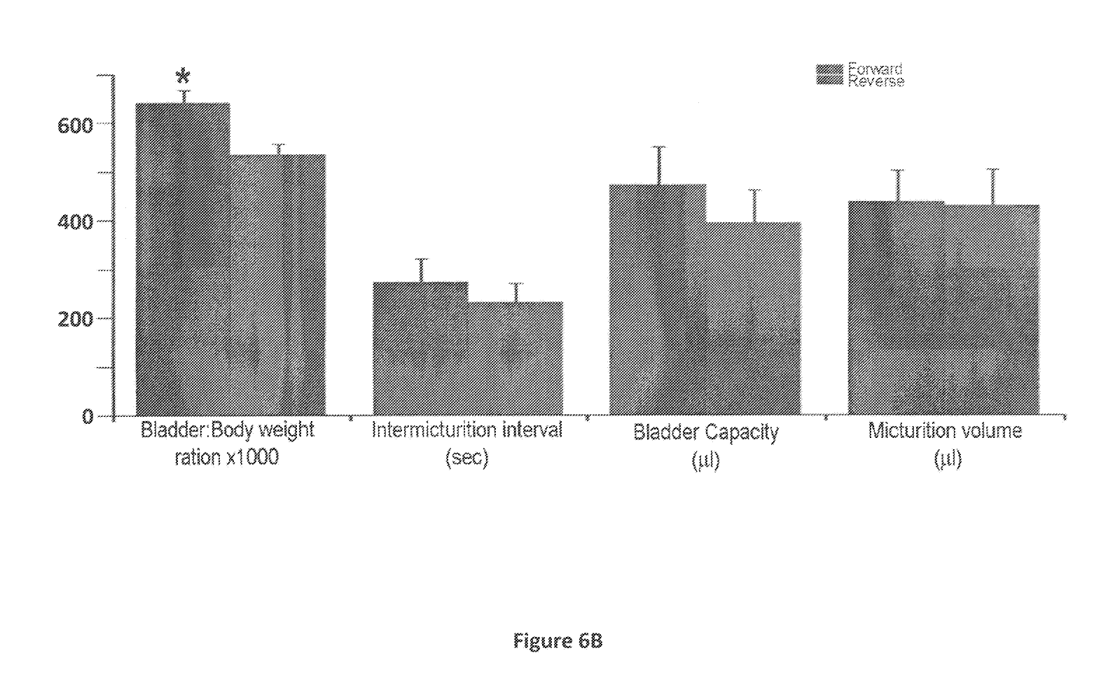

Urodynamics was assessed in rats using in vivo cystometry (n=9 bilateral CRF cDNA and n=14 reverse cDNA). The results are shown in FIG. 6. There was no significant difference in intermicturition interval, bladder capacity (BC), micturition volume (MV), resting pressure, micturition threshold or peak micturition pressure. However, rats injected with AAV-1 expressing CRF cDNA had larger bladder:body weight ratio. The records on the left indicate bladder capacity as the volume of saline infused into the bladder, bladder pressure and micturition volume for a rat injected bilaterally with AAV-1 expressing CRF cDNA. The records on the right show the same endpoints for a rat injected with AAV-1 expressing the reverse sequence. The bars are the mean values for 9 (AAV-1 CRF) and 14 (AAV-1 reverse) rats. *p=0.02.

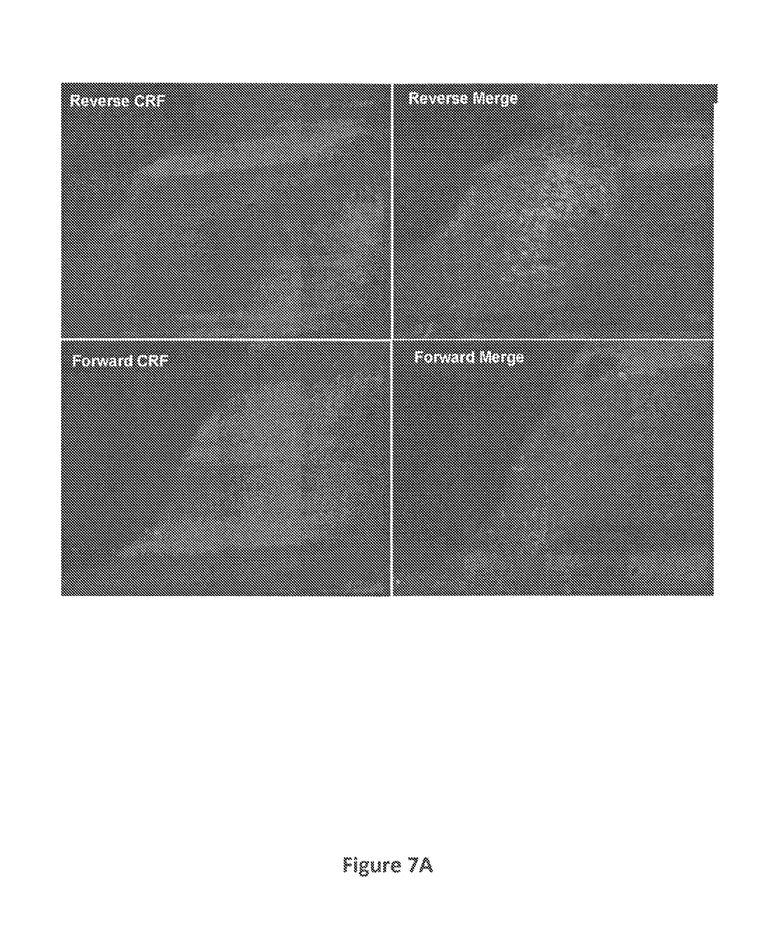

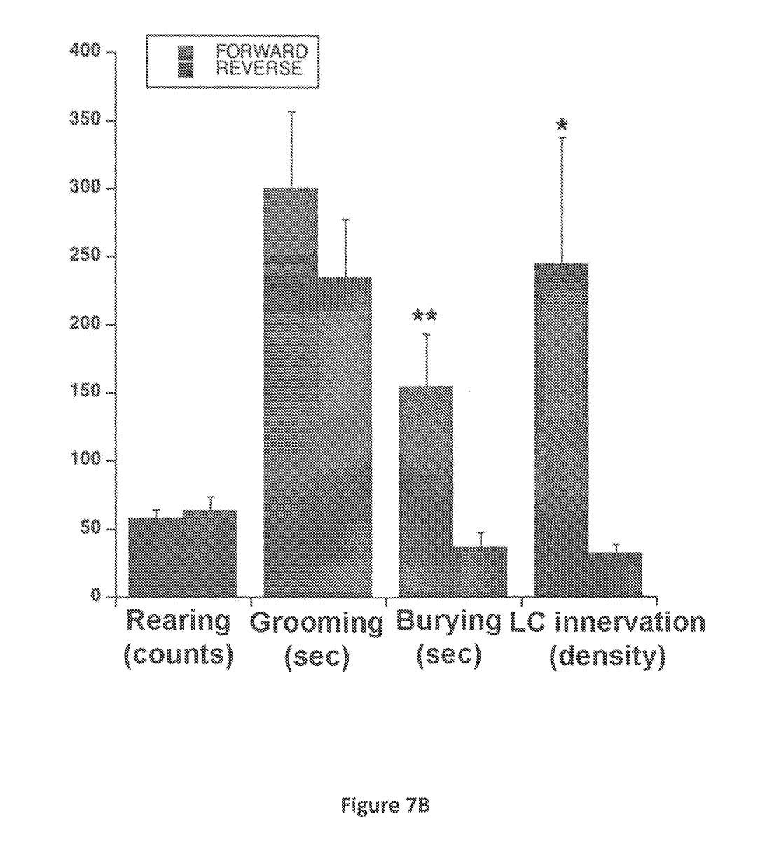

Barrington's nucleus is also a source of CRF afferents to the LC. Fluorescent photomicrographs of CRF innervation of the LC in rats injected with AAV-1 expressing CRF cDNA or AAV-1 expressing the reverse sequence. See FIG. 7. The left panel shows CRF immunoreactivity only and the right panel is the merged image showing the LC as tyrosine hydroxylase immunoreactive neurons (blue). CRF innervation of the LC is denser in rats injected with injected with AAV-1 expressing CRF cDNA as indicated by the bar graph to the right (*p<0.05). The bar graph also indicates a greater incidence of burying, but not rearing or grooming in rats injected with AAV-1 expressing CRF cDNA (n=9) compared to those injected with AAV-1 expressing the reverse sequence (n=12). *p<0.05, **p<0.01.

The data presented herein demonstrate that adeno-associated viral vector technology effectively increases CRF protein expression in Barrington's nucleus neurons. The protein product of the vector is transported from Barrington's nucleus neurons to terminals in the lumbosacral spinal cord by 4 weeks after injection. Although transduction of the CRF gene in Barrington's nucleus neurons did not significantly alter urodynamics as indicated by cystometry, the statistically significant increase in bladder:body weight ratio is consistent with the promotion of urinary retention. Transduction of the CRF gene in Barrington's nucleus neurons enhances CRF innervation of the locus coeruleus. This is sufficient to promote a behavioral endpoint of locus coeruleus activation, i.e., burying. Adeno-associated viral vector technology is effective in manipulating CRF levels in brain neurons. This approach may be used to advantage in patients to enhance bladder control and treat incontinence.

What is claimed is:

1. A vector comprising a corticotropin-releasing factor (CRF) encoding nucleic acid.

2. The vector of claim 1, wherein said CRF has the sequence of SEQ ID NO: 1.

3. The vector of claim 2 which is an adeno-associated viral (AAV) vector.

4. The AAV vector of claim 3 selected from the group of AAV vectors consisting of AAV 1, AAV2, AAV3, AAV4, AAV5, AAV6, AAV7, AAV8, and AAV9.

5. The AAV vector of claim 4, wherein said AAV vector is AAV9.

6. The AAV vector of claim 5, wherein said nucleic acid is operably linked to a promoter which functions in Barrington's nucleus.

7. A composition comprising the vector of claim 6 in a biologically acceptable carrier.

8. A method of treating urinary incontinence in a subject in need of such treatment, said method comprising administering to said subject an effective amount of the composition of claim 7 to said subject, expression of said vector in the cells of said subject being effective to increase urinary retention.

9. The method according to claim 8, in which the urinary incontinence is urge incontinence, stress incontinence, mixed urge/stress incontinence or neurogenic incontinence.

10. The method of claim 8, wherein said composition is administered intravenously.

11. The method of claim 8, wherein said composition is administered directly into Barrinton's nucleus neuronal cells.

12. The method of claim 8, wherein said vector is administered in combination with an immunosuppressive agent.

13. The method of claim 8, wherein said vector is administered in combination with an agent useful for the treatment of incontinence.

14. A kit comprising the AAV vector composition of claim 7 and instructional materials for delivery of said vector to a subject in need thereof.


SEQUENCE LISTING (1 sequence)

SEQ ID NO: 1 - protein, 41 aa, Homo sapiens
SQEPPISLDLTFHLLREVLEMTKADQLAQQAHSNRKLLDIA